(19) United States
(12) Patent Application Publication
(10) Pub. No.: US 2019/0167622 A1
(43) Pub. Date: Jun. 6, 2019

(54) CITRULLINE-CONTAINING FERMENTED MILK AND METHOD FOR PRODUCING SAME

(71) Applicant: Meiji Co., Ltd., Tokyo (JP)

(72) Inventors: Satoshi Takasugi, Odawara-shi, Kanagawa (JP); Yoshie Saito, Odawara-shi, Kanagawa (JP); Yoshitaka Kawai, Odawara-shi, Kanagawa (JP); Keisuke Furuichi, Odawara-shi, Kanagawa (JP); Masashi Nagata, Odawara-shi, Kanagawa (JP); Hiroyuki Itou, Odawara-shi, Kanagawa (JP)

(73) Assignee: Meiji Co., Ltd., Tokyo (JP)

(21) Appl. No.: 16/323,814

(22) PCT Filed: Aug. 8, 2017
(86) PCT No.: PCT/JP2017/028724

§ 371 Date: Feb. 7, 2019

(30) Foreign Application Priority Data
Aug. 9, 2016 (JP) 2016-156663

(51) Int. Cl. A61K 31/198; A23C 9/13; A23L 33/175; A61K 9/00; A61K 35/20; A61P 25/22

(52) U.S. Cl. CPC A61K 31/198; A23C 9/1322; A23L 33/175; A23V 2002/00; A61K 35/20; A61P 25/22; A61K 9/0056

(57) ABSTRACT

The object of the present invention is to provide: a method for producing a citrulline-containing fermented milk; and a citrulline-containing fermented milk produced by the method. The method can prevent milk quality from deteriorating, even when citrulline is added, and the citrulline-containing fermented milk has the effect of improving blood flow. The present invention pertains to: a method for producing a citrulline-containing fermented milk, the method comprising heat-treating a raw material mix that contains citrulline, and fermenting the heat-treated raw material mix, wherein the content of citrulline in the raw material mix that is heat-treated is 0.1-4%; and a fermented milk produced by the method.

### TECHNICAL FIELD

The present invention relates to citrulline-containing fermented milk and a method for producing the same.

### BACKGROUND ART

Citrulline is a functional amino acid; in Japan, it was first approved for use as a medicine, then in 2007 approved for use as food. In overseas, citrulline has been taken in as food on or before 2007, and has been sold as a supplement due to its function of improvement of blood flow, prevention of arteriosclerosis, and increase in energy level. In addition, citrulline has been reported to have other functions such as improvement of sensitivity to cold (“hie-sho”), improvement of skin function, alleviation/recovery of fatigue, muscle growth, improvement of motor function, etc.

Concrete examples of such reports include: a case wherein blood arginine concentration is raised promptly by containing citrulline and arginine as active ingredients in an oral agent, thereby attempting to increase blood flow (Patent Document 1), and a case wherein prevention and improvement of bone loss are attempted by using citrulline as an active ingredient (Patent Document 2).

On the other hand, when citrulline is used for foods and drinks, there is a problem that the taste and smell of foods and drinks will change, which must be dealt with. For example, the following attempts have been made: bitterness of citrulline itself is masked by using a thickener and γ-PGA (Patent Document 3), and the offensive smell generated during heating is suppressed by blending an acidulant or a pH adjuster into a citrulline-containing food or drink and by adjusting the pH to an acidic range (Patent Document 4). However, in the field of foods, there are practically no examples of specific products in which these problems are sufficiently satisfactorily solved.

### CITATION LIST

### Patent Document

### [Patent Document 1] WO 2009/048148

### [Patent Document 2] JP A 2012-67062

### [Patent Document 3] JP A 2009-118743

### [Patent Document 4] JP A 2009-171860

### [Patent Document 5] WO 2015/111597

### SUMMARY OF INVENTION

### Problems to be Solved by Invention

Meanwhile, the inventors of the present invention focused on the usefulness of citrulline, and from the viewpoint of being capable of providing functional foods, etc. which are extremely useful for maintenance and promotion of people's health by means of application of citrulline in the field of fermented milk such as yogurt, the inventors have examined citrulline-containing yogurt produced using citrulline high-yield lactic acid bacteria (Patent Document 5). Since such yogurt basically need not go through a heat sterilization step after citrulline production, it is superior in that it can avoid problems such as deterioration of color tone, smell, and flavor; however, there is a problem that adjustment of the citrulline concentration in yogurt is not always easy. In order to solve this problem, it is necessary to adjust by adding citrulline from the outside; however, in this case as well, a heat sterilization step is required, so it is predicted that problems of deterioration of color tone, smell, and flavor cannot be avoided. Accordingly, an object of the present invention is to provide a method for rationally producing yogurt which solves all the above problems, has no problem of deterioration of color tone, smell, and flavor and has useful functions such as an effect of improving blood flow and the like, as well as to provide yogurt which is produced by such method.

### Means of Solving Problems

The inventors of the present invention have keenly studied to solve the above-mentioned problems and found that, by adjusting the citrulline content in a raw material mix to a predetermined range, upon heat sterilization, deterioration of color tone, smell, and flavor can be prevented without adding additives such as a thickener and a pH adjusting agent, and the obtained citrulline-containing fermented milk exerts useful effects such as remarkable improvement in blood flow; and as a result of further research, the inventors have completed the present invention.

That is, the present invention relates to a method for producing citrulline-containing fermented milk and fermented milk produced by said method.

[1] A method for producing citrulline-containing fermented milk, comprising heat-treating a raw material mix containing citrulline, and fermenting the heat-treated raw material mix, wherein the citrulline content in the raw material mix to be heat treated is 0.1 to 4%.
[2] The method according to the above [1], wherein the raw material mix comprises citrulline derived from citrulline-producing microorganisms.
[3] A fermented milk produced by the method according to the above [1] or [2].
[4] The fermented milk according to the above [3], wherein the fermented milk is yogurt.
[5] The fermented milk according to the above [3] or [4], wherein the citrulline content in the fermented milk is 0.3 to 0.5%.
[6] The fermented milk according to any one of the above [3] to [5], which has one or more uses selected from the group consisting of improvement of blood flow, improvement of sensitivity to cold, improvement of skin quality, and normalization of mental condition.

### Advantageous Effects of Invention

Citrulline is generally known to be difficult to apply to foods, because bitterness of citrulline itself and offensive smell generated upon heating are considered to be problematic, and it is said that citrulline can only be used at low concentrations (Patent Document 4 and others). Whereas in the application to fermented milk, unexpectedly, even in the case where citrulline at a relatively high concentration is contained in a raw material mix at the time of heating, there is no problem in terms of color tone, smell and flavor. Therefore, according to the present invention, it is possible to adjust the citrulline concentration freely within the range of its effective concentration, and to provide citrulline-containing fermented milk which is comparable to conventional fermented milk in terms of color tone, smell, flavor and the like, and which sufficiently exhibits the effect of citrulline such as blood-flow improving effect. Such citrulline-containing fermented milk can be adjusted so as to exert effects such as improvement of sensitivity to cold, improvement of skin quality and normalization of mental condition, in addition to blood-flow improving effect.

### BRIEF DESCRIPTION OF DRAWINGS

FIG. 1 shows changes in the amount of blood flow change before and after cold water loading at the end of the test, measured in Example 1.

FIG. 2 shows the results of VAS of “coldness of the body” in each group before and after the test, measured in Example 1.

FIG. 3 shows the results of VAS of “body glowing” in each group before and after the test, measured in Example 1.

FIG. 4 shows the results of VAS of “moist feeling of the skin” in each group before and after the test, measured in Example 1.

FIG. 5 shows the results of VAS of “dry feeling of the skin” in each group before and after the test, measured in Example 1.

FIG. 6 shows the results of POMS (T score) of “tension-anxiety” in each group before and after the test, measured in Example 1.

FIG. 7 shows the results of POMS (T score) of “anger-hostility” in each group before and after the test, measured in Example 1.

FIG. 8 shows the results of POMS (T score) of “depression-dejection” in each group before and after the test, measured in Example 1.

FIG. 9 shows the results of POMS (T score) of “liveliness” in each group before and after the test, measured in Example 1.

FIG. 10 shows the results of POMS (T score) of “fatigue” in each group before and after the test, measured in Example 1.

FIG. 11 shows the results of POMS (T score) of “confusion” in each group before and after the test, measured in Example 1.

### EMBODIMENTS FOR CARRYING OUT INVENTION

In general, fermented milk is produced by preparing a raw material mix which is raw material of the fermented milk, sterilizing the prepared raw material mix by heat treatment, inoculating lactic acid bacteria to the sterilized raw material mix, then fermenting it.

The method for producing citrulline-containing fermented milk of the present invention comprises heat-treating a raw material mix containing citrulline and fermenting the heat-treated raw material mix.

The content of citrulline in the raw material mix to be heat treated may be 0.1 to 4%, preferably 1 to 4%, and particularly preferably 2 to 4%. When the citrulline content is low, effects such as blood flow improving action tend to be difficult to obtain, and when the citrulline content is high, color tone, smell, flavor and the like of the produced fermented milk tend to deteriorate.

“Fermented milk” according to the present invention is a dairy product and a processed product obtained by fermenting milk, etc. with lactic acid bacteria or yeast, etc., and includes “fermented milk”, “dairy product fermented milk drink” and “fermented milk drink” and the like defined by the Ministerial Ordinance on Milk and Milk products Concerning Compositional Standards, etc. The fermented milk produced according to the present invention may be, for example, yogurt, cheese, cream cheese, etc. The fermented milk produced according to the present invention may be either pre-fermentation type yogurt wherein a raw material mix is fermented in a tank, etc., then filled in a container; or post-fermentation type yogurt wherein a raw material mix is filled in a container, then fermented. The fermented milk produced according to the present invention may be, for example, plain yogurt, set type yogurt (solid fermented milk), soft yogurt (pasty fermented milk), and drink yogurt (liquid fermented milk) and the like. In the production method of the present invention, since fermented milk having sufficient hardness can be produced, it can be suitably utilized for set type yogurt.

The “raw material mix” according to the present invention includes milk, milk constituents, or a composition comprising milk constituents. Milk constituents include, for example, raw milk, cow's milk, skimmed milk, whole milk powder, skimmed milk powder, full fat concentrated milk, concentrated skimmed milk, sweetened condensed milk, sweetened condensed skimmed milk, evaporated milk, evaporated skimmed milk, whey, whey powder, desalted whey, desalted whey powder, whey protein concentrate (WPC), whey protein isolate (WPI), α-lactalbumin, β-lactoglobulin, milk protein concentrate (MPC), casein, sodium caseinate, calcium caseinate, cream, fermented cream, compounded cream, cream powder, butter, fermented butter, buttermilk, buttermilk powder and butter oil etc. The raw material mix may contain two or more kinds of milk constituents. In addition to milk constituents, the raw material mix may further contain, for example, water, lipids, proteins, sugars, flavoring components, flavors, pigments, minerals (salts), vitamins and other additives for foods, etc. The raw material mix may also contain a gelatin liquid, etc. previously dissolved by heating.

Citrulline which can be used in the present invention is not particularly limited. Citrulline produced by plants and microorganisms or citrulline obtained by chemical synthesis may be used.

When citrulline is added to the raw material mix according to the present invention, those which have been concentrated, refined or extracted from a natural product or chemically synthesized may be added; for example, citrulline-containing culture produced using citrulline-producing microorganisms can also be added to the raw material mix.

Here, the citrulline-producing microorganism is not particularly limited as long as it produces citrulline, but is preferably a lactic acid bacterium having the ability to produce citrulline.

Lactic acid bacteria having citrulline-producing ability are preferably one or more kinds selected from the group consisting of Lactococcus lactis, Lactobacillus fermentum, Lactobacillus buchneri, Enterococcus faecalis, Oenococcus oeni, Pediococcus pentosaceus, Lactobacillus amylovorus, Lactobacillus brevis, and Lactobacillus reuteri; and more preferably, they are Lactococcus lactis.

Lactococcus lactis having citrulline-producing ability of the present invention is preferably one or more kinds selected from the group consisting of Lactococcus lactis subspecies lactis and Lactococcus lactis subspecies cremoris; and more preferably, Lactococcus lactis subspecies lactis.

For heat sterilization of raw material mix, high temperature/short time (HTST) treatment or ultra-high temperature (UHT) treatment can be used. Therefore, since the same sterilization conditions as milk and milk drinks, etc. can be set, it is possible to use the same method and equipment as the products such as milk and milk drinks in the heat sterilization of raw material mix, and the production efficiency of various products is not lowered in a factory of dairy industry as a whole and new installation of equipment for each kind of products is not required.

The temperature at which the raw material mix is sterilized is, for example, 63° C. or higher, preferably 72° C. or higher, more preferably 115° C. or higher, still more preferably 116° C. or higher, still more preferably 120° C. or higher, and still more preferably 125° C. or higher. The temperature at which the raw material mix is sterilized is preferably 150° C. or less, more preferably 145° C. or less, and still more preferably 140° C. or less.

The time for sterilizing the raw material mix is preferably from 1 second to 30 minutes, more preferably from 1 second to 300 seconds, still more preferably from 1 second to 120 seconds, still more preferably from 1 second to 60 seconds, still more preferably 1 second to 30 seconds, and most preferably 1 second to 10 seconds.

The combination of the temperature and the time of the heat sterilization is, for example, a normalized or widely used heat sterilization condition, which may be 72° C. to 75° C. for 15 seconds (HTST), or 120° C. to 150° C. for 1 second to 5 seconds (UHT).

In the present invention, preparation of a raw material mix can be carried out by at first mixing all raw materials except citrulline, adding citrulline to the mixed raw materials, then conducting heat sterilization, but it is not limited thereto. For example, a raw material mix can be prepared by mixing citrulline with a part of raw materials requiring heat sterilization, heat sterilizing the obtained mixture, and mixing with separately-prepared and sterilized remaining raw materials. Therefore, in the present specification, “raw material mix” includes not only a prepared raw material mix just before fermentation but also a mixture of a part of raw materials and citrulline, which is prepared just before heat treatment.

For example, for the addition of citrulline, when a culture of citrulline-producing microorganisms is added, then the raw material mix may be prepared with sterilization by heat treatment so that the added citrulline-producing microorganisms do not affect fermentation, or the citrulline content does not become excessive due to the continuance of citrulline production.

The citrulline-containing fermented milk produced by the production method of the present invention has a quality comparable to conventional fermented milk in terms of color tone, smell, flavor and the like. In such citrulline-containing fermented milk, the citrulline content may be the same as the citrulline content in the raw material mix, or it may be diluted by a raw material added after the heat treatment. Therefore, the citrulline content in the fermented milk of the present invention is 0.1 to 4%, preferably 0.1 to 1%, particularly preferably 0.3 to 0.5%.

Rather than citrulline alone, the citrulline-containing fermented milk of the present invention can be used to improve body conditions, such as for improving blood flow, for improving sensitivity to cold, for improving skin quality.

In addition, rather than citrulline alone, the citrulline-containing fermented milk of the present invention can be used for applications such as normalizing the mental condition, in particular relieving tension and anxiety, sedation of anger and hostility, and improving depression and dejection, recovery of fatigue and sedation of confusion.

### EXAMPLES

Hereinafter, the method according to the present invention will be described in more detail based on examples and test examples, but the present invention is not limited thereto.

### [Example 1] Effect of Citrulline-Containing Yogurt of Improving Blood Flow

### (a) Cold Water Loading Test

Women in their thirties satisfying the diagnostic criteria of sensitivity to cold (Terasawa, 1987, Pharmacology Journal, 41: 85-96, in Japanese) were divided into three groups ((i) water group, (ii) citrulline water (Cit-water) group, (iii) yogurt containing citrulline (CitYo) group), and an open parallel-group comparison study was conducted.

Subjects in each group were asked to take one test meal, i.e., 180 mL/day of water, Cit-water (Cit blended: 800 mg/bottle), and CitYo (Cit blended: 800 mg/bottle) during the period from dinner to bedtime for 14 days. Prior to the ingestion of each test meal (water, Cit-water, CitYo), the test meal was warmed to a temperature close to human skin using a microwave oven. After ingesting each test meal for 14 days, a cold water loading test was conducted.

With respect to the citrulline, the following solutions were mixed and stirred under cooling and used: a solution wherein raw material cream, concentrated skimmed milk and raw water were mixed and dissolved was heat sterilized at 110° C. for 30 seconds, cooled to 43° C., to which lactic acid bacteria starter was added and fermented to an acidity of 0.75, then homogenized; a solution wherein raw water, pectin, sucralose, citrulline and flavor were mixed and dissolved, then heat sterilized at 80° C. for 10 minutes; and a solution wherein raw water and sugar were mixed and dissolved, then heat-sterilized at 80° C. for 10 minutes. The blending ratios of the raw materials are shown in Table 1.

TABLE 1
Blending ratios of raw materials ot citrulline yogurt.

| Raw material | Blending ratio (%) |
|---|---|
| Raw material cream | 1.008 |
| Skimmed milk powder | 24.57 |
| Lactic acid bacteria starter | 1.2 |
| Sugar | 2.5 |
| Citrulline | 0.45 |
| Sucralose | 0.0064 |
| Pectin | 0.25 |
| Flavor | 0.03 |
| Raw material water | 70.0 |
| Total | 100 |

Average±standard deviation of the age and BMI of the subjects are shown in Table 2.

TABLE 2
Average ± standard deviation of age and BMI of subjects.

| | | Water group | Cit-water group | CitYo group |
|---|---|---|---|---|
| | n | 9 | 10 | 10 |
| Age | (years old) | 35.9 ± 3.1 | 35.4 ± 3.7 | 35.9 ± 3.7 |
| BMI | kg/m² | 21.0 ± 2.5 | 20.8 ± 2.4 | 21.9 ± 3.0 |

In the cold water loading test, subjects in each group were fasted for 5 hours or more prior to the start of the cold water loading test, worn with a predetermined examination cloth, and they stayed and rested in a room with constant temperature and constant humidity (room temperature: 25±1° C., humidity: 50±10%) for about 30 minutes. Subsequently, one hour after ingesting one bottle (180 ml) of respective test meal, the subjects in each group immersed their right hand in cold water (20° C.) for 1 minute (cold water loading). Thereafter, the subjects in each group gently wiped the moisture on the right hand, and the blood flow on the right hand was continuously monitored using a laser two-dimensional blood flow imaging device (OMEGAZONE OZ-1, Omega Wave Company) from 2 minutes after the cold water loading (i.e., immediately after the cold water loading) to 21 minutes after. For the blood flow rate, the average value was calculated every 1 minute, and expressed as an amount of change from the value just after the cold water loading which was set to be 0. Regarding VAS (Visual Analog Scale) method, which is a pain assessment method, the average value for two and half weeks until three days before the start of ingestion of test meal is set as “pre value”, and the average value over three days from the 12th day to 14th day after the start of ingestion of test meal is set as “14 day value”.

### (b) Psychological Examination

Regarding the psychological examination: POMS brief form (Profile of Mood States—Brief Form Japanese Version), examination was conducted about three weeks before the start of the test (“pre value”) and immediately after the end of the test (“14 day value”). Concretely, with respect to 6 items of “tension-anxiety”, “depression-dejection”, “anger-hostility”, “liveliness”, “fatigue” and “confusion”, we referred to the T-score conversion table of women aged 30-39 years described in “POMS brief form, guidance and explanation of cases” (Ed. Kazuhito Yokoyama, Kaneko Shobo) to obtain and analyze T scores.

### (c) Statistical Analysis

In the significant difference test of the age and mean value of BMI of subjects, Tukey's multiple comparison test was used after homogeneity of variance was confirmed by the Bartlett method.

In the significant difference test of the average value of changes in the amount of blood flow change, after homogeneity of variance was confirmed by the Bartlett method at each time, when homogeneity of variance was confirmed, Dunnett's multiple comparison test was used, and when homogeneity of variance was not confirmed, Steel's multiple comparison test was used with the water group as a control. The significance level was 5% on both sides.

In the significance test of the average value of VAS, after homogeneity of variance was confirmed by F test for pre value and 14 day value in each group, when homogeneity of variance was confirmed, paired student's t-test was used, and when homogeneity of variance was not confirmed, Wilcoxon signed rank test was used. In the significance test of the average value of POMS, the Wilcoxon signed rank test was used for the pre value and the 14 day value in each group.

### (d) Test Result

As a result of the cold water loading test, it was confirmed that peripheral blood flow improving effect was high in citrulline-containing yogurt (CitYo) as compared with citrulline water (Cit-water, citrulline alone) (FIG. 1).

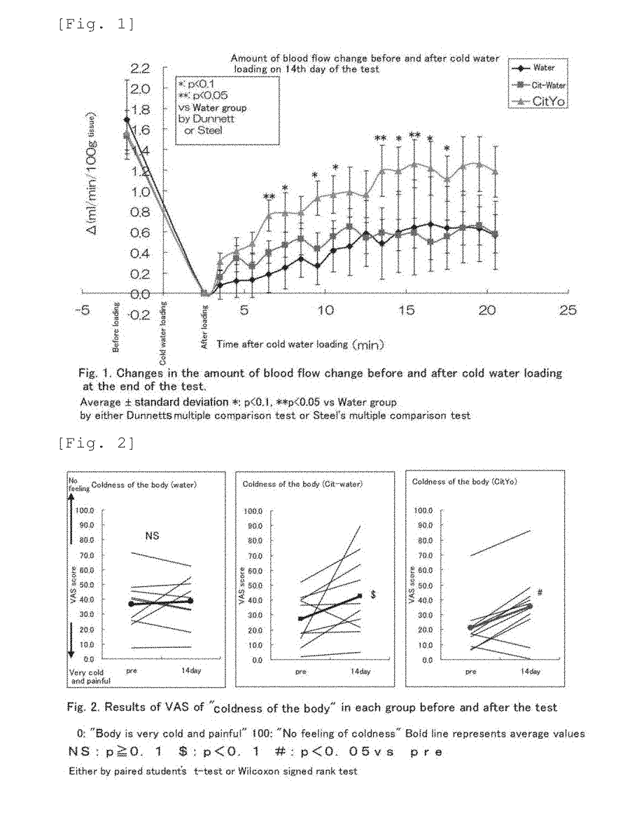

As a result of psychological examination, it was confirmed that the effect of improving sensitivity to cold can be actually felt with CitYo (FIG. 2, FIG. 3).

Incidentally, in yogurt without citrulline, blood flow improving effect was not confirmed (data not shown).

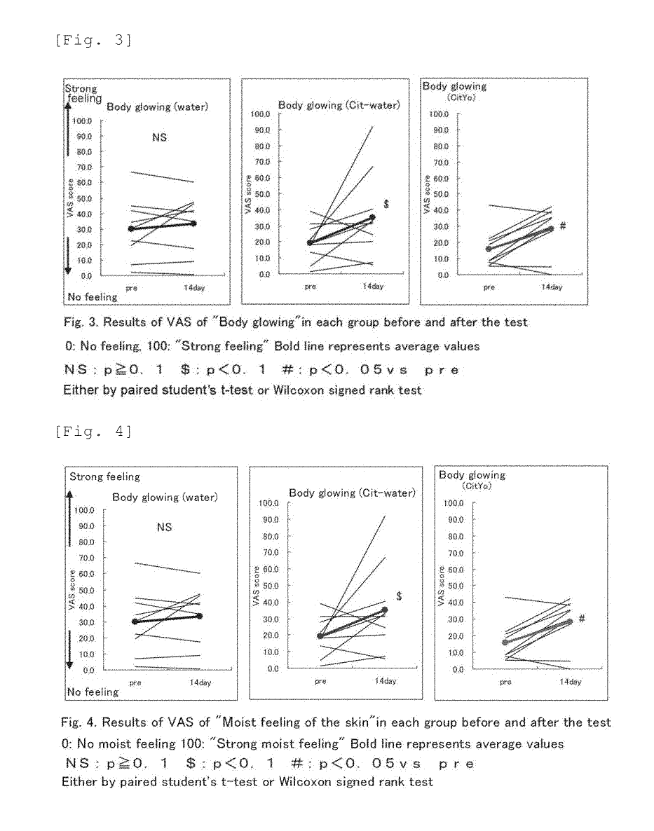

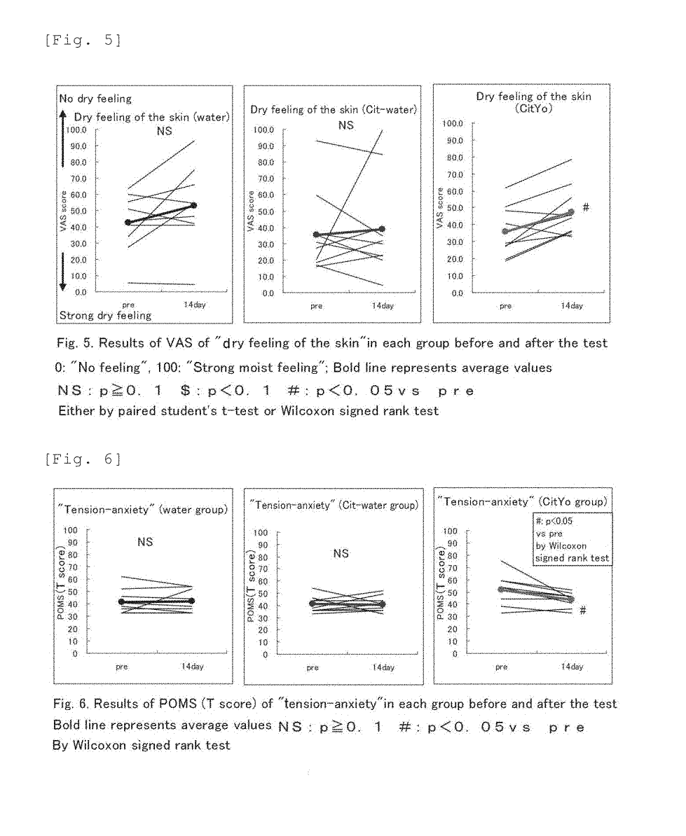

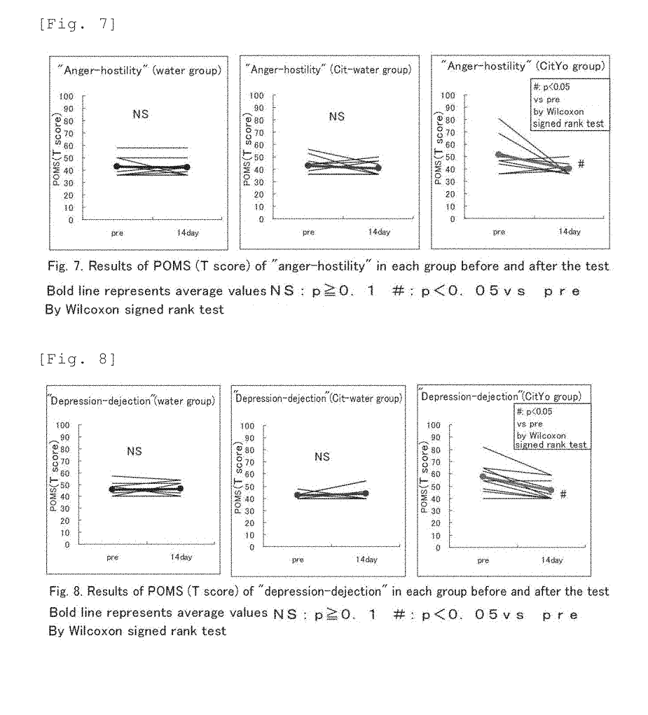

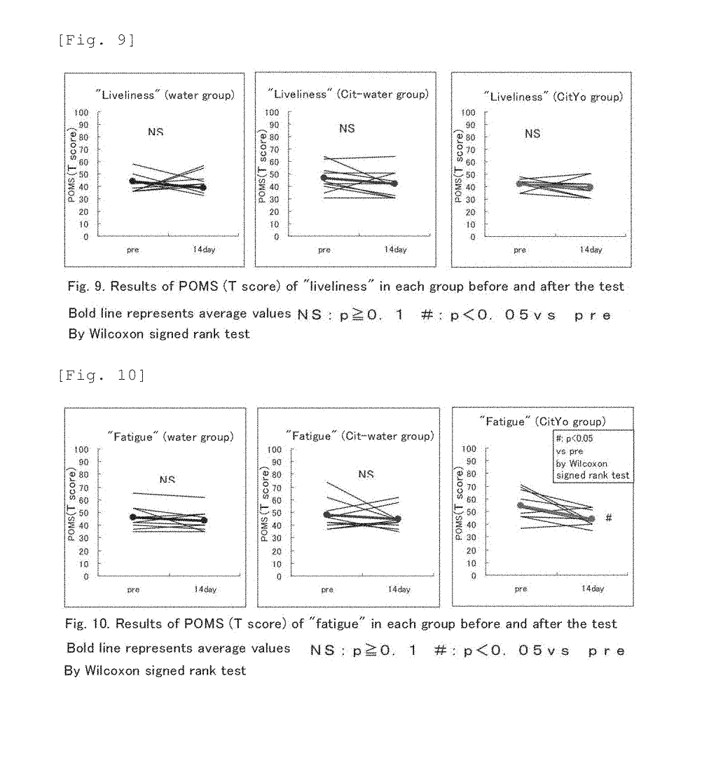

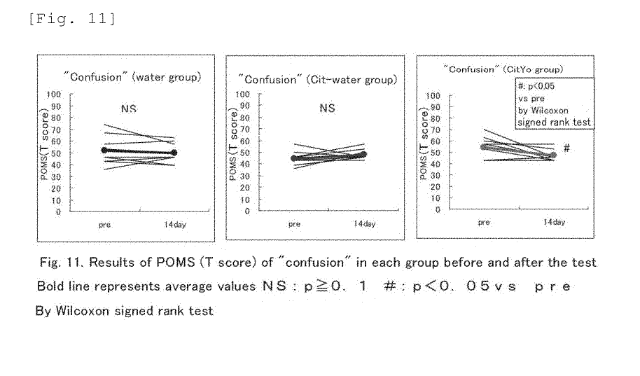

In addition to the effect of improving blood flow, it was also confirmed that CitYo also shows the effect of improving skin quality (FIG. 4, FIG. 5) and the effect of normalizing the mental condition (FIGS. 6 to 11).

### [Example 2] Change in Citrulline-Containing Reduced Skimmed Milk after Heat Treatment

To 10% reduced skimmed milk solution, citrulline (a commercial product of reagent was purchased) was added with adjusting its concentration to 0%, 2%, 4% and 8%, then these solutions were heat-sterilized at 121° C. for 15 minutes. Sensory tests were conducted using the citrulline-containing raw material mixes of the respective concentrations obtained.

In the sensory tests, color tone, smell and flavor were evaluated by three panelists, and the presence or absence of precipitate was also confirmed. The results are shown in Table 3.

TABLE 3
Change in citrulline-containing reduced
skimmed milk after heat treatment.

| Citrulline | Presence/ | | | |
|---|---|---|---|---|
| concentration | absence of | | | |
| (%) | precipitate | Color tone | Smell | Flavor |
| 0 | None | Light brown | Normal | Normal |
| 2 | None | Light brown | Slightly | Slightly |
| | | | bad | bad (weak |
| | | | (burning | bitterness) |
| | | | smell) | |
| 4 | None | Brown | Bad | Bad |
| | | | (burning | (bitterness) |
| | | | smell) | |
| 8 | Small amount | Dark brown | Very bad | Very bad |
| | | | (strong | (strong |
| | | | burning | bitterness) |
| | | | smell) | |

From the results shown in Table 3, when citrulline is used in a raw material mix, it is preferable to set the citrulline concentration to 4% or less, and if it is 2% or less, then the citrulline does not show almost any problem from the viewpoint of heat resistance, and smell and flavor of the fermented milk produced.

### INDUSTRIAL APPLICABILITY

The present invention provides a production method of citrulline-containing fermented milk, which prevents deterioration of the quality of a raw material mix occurred during the production, as well as citrulline-containing fermented milk produced by said method which has effects such as remarkable improvement of blood flow, etc.

1. A method for producing citrulline-containing fermented milk, comprising heat-treating a raw material mix containing citrulline, and fermenting the heat-treated raw material mix, wherein the citrulline content in the raw material mix to be heat treated is 0.1 to 4%.

2. The method according to claim 1, wherein the raw material mix comprises citrulline derived from citrulline-producing microorganisms.

3. A fermented milk produced by the method according to claim 1.

4. The fermented milk according to claim 3, wherein the fermented milk is yogurt.

5. The fermented milk according to claim 3, wherein the citrulline content in the fermented milk is 0.3 to 0.5%.

6. The fermented milk according to claim 3, which has one or more uses selected from the group consisting of improvement of blood flow, improvement of sensitivity to cold, improvement of skin quality, and normalization of mental condition.
